(19) United States
(12) Patent Application Publication
(10) Pub. No.: US 2020/0082923 A1
(43) Pub. Date: Mar. 12, 2020

(54) SYSTEMS AND METHODS OF MONITORING MEDICATION COMPLIANCE

(71) Applicants: Sean Harrison, Cary, NC (US); Brian Dalgetty, Cary, NC (US)

(72) Inventors: Sean Harrison, Cary, NC (US); Brian Dalgetty, Cary, NC (US)

(21) Appl. No.: 16/565,383

(22) Filed: Sep. 9, 2019

Related U.S. Application Data
(60) Provisional application No. 62/728,693, filed on Sep. 7, 2018.

(51) Int. Cl. G16H 20/10; G06N 3/02; G16H 40/67

(52) U.S. Cl. CPC G16H 20/10; G16H 40/67; G06N 3/02

(57) ABSTRACT

Systems and methods of monitoring medication compliance are provided. A first node receives from a second node a picture of a pill bottle label and a picture of the number of pills in the pill bottle. The information on the label is predicted from the picture of the pill bottle. The medication and number of pills is predicted from the picture of the number of pills. Information associated with compliance to the dosage regimen of the medication is determined based on the predicted number of pills and the dosage regimen. The second node sends to the first node an indication of the information associated with the medication compliance.

### CROSS REFERENCE TO RELATED APPLICATIONS

This application claims the benefit of U.S. Provisional App. No. 62/728,693, filed Sep. 7, 2018, the contents of which are incorporated herein by reference in its entirety.

### FIELD OF DISCLOSURE

The present disclosure relates generally to the field of medication compliance, and in particular to systems and methods of monitoring medication compliance.

### BACKGROUND

Medication non-adherence is a major problem in healthcare. There are more and more complex chronic patients as baby boomers age. Further, fifty percent of all patients have more than one chronic disease. Also, fifty percent of all patients have poor rates of medication adherence. Indeed, ten percent of all hospital admissions are caused by medication non-adherence.

Medication non-adherence is a complex, expensive process. Every home health and hospice visit requires a comprehensive medication inventory, and there are thirty million annual inventories. An inventory is a manual process, requiring the transcription of labels, counting pills and manually entering data in the electronic health record (EHR), etc. The process is error-prone typically from dealing with small print, cryptic medication names and time pressures. Further, the inventory process is time consuming typically averaging forty five minutes per typical Medicare patient. The inventory process is also tedious, frustrating and unenjoyable for nurses.

A prescription label has a large amount of information that must be inventoried and entered in the EHR. This information includes the patient name, the manufacturer/brand name, form (e.g., 10 mg tablets), the generic medication name, the directions, the expiration date, refills, physician name, physician phone number, quantity, prescription number, date filled, pharmacy name and address, pharmacy phone number and a barcode. Then, the pills have to be counted.

Accordingly, there is a need for improved techniques to monitor medication compliance. In addition, other desirable features and characteristics of the present disclosure will become apparent from the subsequent detailed description and embodiments, taken in conjunction with the accompanying figures and the foregoing technical field and background.

The Background section of this document is provided to place embodiments of the present disclosure in technological and operational context, to assist those of skill in the art in understanding their scope and utility. Unless explicitly identified as such, no statement herein is admitted to be prior art merely by its inclusion in the Background section.

### SUMMARY

The following presents a simplified summary of the disclosure in order to provide a basic understanding to those of skill in the art. This summary is not an extensive overview of the disclosure and is not intended to identify key/critical elements of embodiments of the disclosure or to delineate the scope of the disclosure. The sole purpose of this summary is to present some concepts disclosed herein in a simplified form as a prelude to the more detailed description that is presented later.

Briefly described, embodiments of the present disclosure relate to systems and methods for monitoring medication compliance. According to one aspect, a method of monitoring medication compliance, includes: receiving, by a first node, from a second node, a first image of a medication label having information that indicates at least a medication and a dosage regimen of the medication. The medication label is disposed on a medication container. Information displayed on the label is predicted based on the first image. The first node receives, from the second node, a second image of one or more pills distributed on a surface, where the one or more pills represents all of the pills disposed in the container when distributed on the surface, and the medication based on the second image is predicted so that visual characteristics of the predicted medication are obtained. A number of pills displayed in the second image is predicted based on the predicted medication and the visual characteristics, and information associated with compliance to the dosage regimen of the medication is determined based on the predicted number of pills and the dosage regimen. The first node sends, to the second node, an indication of the information associated with the medication compliance.

According to another aspect, the second node is a wireless device such as a smartphone, a tablet, a smartwatch, a telehealth or a hand-held device. Communication between the first node and the second node is via the internet. The first node includes a receiver circuit, a label information prediction circuit, a pill count prediction circuit, a patient records database, a medication compliance circuit and a transmitter circuit. The indication of the information associated with the medication compliance is displayed on a screen of the second node. The first image is processed by an image preprocessor circuit, a feature extraction circuit, label classification neural networks, and a label prediction circuit. The label prediction circuit outputs predicted label information to an anonymize circuit, which transmits the patient identifier and the predicted label information to a patient records database. The second image is processed by an image preprocessor circuit and a feature extraction circuit, which outputs isolated pill images to pill identification neural networks and a pill identity judgement circuit, which outputs the predicted pill to a pill visual characteristics circuit, which compares the predicted pill with data from a pill visual characteristics database, and the pills image is processed by a pill counter neural networks and a pill judgement circuit, to output a predicted pill count and the predicted pill to a medication compliance circuit, which interacts with the patient records database to output the information associated with the medication compliance.

According to another aspect, a wound evaluation module is disclosed that includes: recording an image of the wound with the second node, and transmitting wound information to the first mode. The wound information is compared with previous pictures of the wound and wound information in a wound database. The first node sends information about wound healing.

According to another aspect, a first node includes: at least one processor and a memory, the memory comprising instructions executable by the at least one processor whereby a wireless device is configured to: receive, from a second node, a first image of a medication label having information that indicates at least a medication and a dosage regimen of the medication, wherein the medication label is disposed on a medication container. The first node is configured to predict the information displayed on the label based on the first image, and receive, from the second node, a second image of one or more pills distributed on a surface, where the one or more pills represents all of the pills disposed in the container when distributed on the surface. The second image is analyzed to predict the medication based on the second image and to obtain visual characteristics of the predicted medication. The first node also predicts a number of pills displayed in the second image based on the predicted medication and the visual characteristics, determines determine information associated with compliance to the dosage regimen of the medication based on the predicted number of pills and the dosage regimen, and sends, to the second node, an indication of the information associated with the medication compliance

According to another aspect, a method performed by a second node includes: obtaining a first image of a medication label having information that indicates at least a medication and a dosage regimen of the medication, wherein the medication label is disposed on a medication container, and sending, by the second node, to a first node, the first image. A second image is obtained of one or more pills distributed on a surface, wherein the one or more pills represents all of the pills disposed in the container when distributed on the surface. The second node sends the second image to the first node. The second node receives, from the first node, an indication of compliance to the dosage regimen of the medication based on the first and second images.

According to one aspect, a system for monitoring medication compliance, includes: a first node including a camera, a microphone, an accelerometer and a memory; and a second node including a memory and a processor. The first node is configured to obtain a first of a medication label having information that indicates at least a medication and a dosage regimen of the medication, where the medication label is disposed on a medication container. The first node sends to first image to the second node, and the first node obtains a second image of one or more pills distributed on a surface, where the one or more pills represents all of the pills disposed in the container when distributed on the surface. The second node is configured to predict information on the label based in the first image, predict the medication based on the second image, obtain visual characteristics of the predicted medication, predict a number of pills displayed in the second image based on the predicted medication and the visual characteristics, determine information associated with compliance to the dosage regimen of the medication based on the predicted number of pills and the dosage regimen, and send to the first node an indication of the information associated with the medication compliance.

### BRIEF DESCRIPTION OF THE DRAWINGS

The present disclosure will now be described more fully hereinafter with reference to the accompanying drawings, in which embodiments of the disclosure are shown. However, this disclosure should not be construed as limited to the embodiments set forth herein. Rather, these embodiments are provided so that this disclosure will be thorough and complete, and will fully convey the scope of the disclosure to those skilled in the art. Like numbers refer to like elements throughout.

FIG. 1 illustrates one embodiment of a system of monitoring medication compliance in conformance with various aspects as described herein.

FIG. 2 illustrates one embodiment of a system of predicting medication label information in conformance with various aspects as described herein.

FIG. 3 illustrates one embodiment of a system of predicting a medication pill count in conformance with various aspects as described herein.

FIG. 4 illustrates one embodiment of a system of determining medication compliance in accordance with various aspects as described herein.

FIG. 5 illustrates one embodiment of a node in accordance with various aspects as described herein.

FIG. 6 illustrates another embodiment of a node in accordance with various aspects as described herein.

FIG. 7 illustrates one embodiment of a method of monitoring medication compliance in accordance with various aspects as described herein.

FIG. 8 illustrates another embodiment of a method of monitoring medication compliance in accordance with various aspects as described herein.

FIG. 9 illustrates another embodiment of a node in accordance with various aspects as described herein.

FIG. 10 illustrates an embodiment of an app on a hand held device in accordance with various aspects as described herein.

FIG. 11 illustrates an embodiment of an app on a hand held device photographing a pill bottle in accordance with various aspects as described herein.

FIG. 12 illustrates an embodiment of an app on a hand held device extracting label information from a photograph of a pill bottle in accordance with various aspects as described herein.

FIG. 13 illustrates an embodiment of an app on a hand held device photographing a pill in accordance with various aspects as described herein.

FIG. 14 illustrates an embodiment of an app on a hand held extracting pill information in accordance with various aspects as described herein.

FIG. 15 illustrates an embodiment of an app on a hand held device photographing a plurality of pills in accordance with various aspects as described herein.

FIG. 16 illustrates an embodiment of an app on a hand held device counting a plurality of pills in accordance with various aspects as described herein.

FIG. 17 illustrates an embodiment of an app on a hand held device displaying patient information in accordance with various aspects as described herein.

FIG. 18 illustrates an embodiment of an app on a hand held device displaying menus in accordance with various aspects as described herein.

FIG. 19 Illustrates a wound image evaluation embodiment of the disclosure.

FIG. 20 Illustrates an additional aspect of the wound image evaluation embodiment of the disclosure.

FIG. 21 Illustrates an additional information relaying aspect of a wound image evaluation embodiment of the disclosure.

FIG. 22 Illustrates an additional classification aspect of the wound image evaluation embodiment of the disclosure.

FIG. 23 Illustrates a shared classification aspect of the wound image evaluation embodiment of the disclosure.

FIG. 24 illustrates an embodiment of the wound healing module of the disclosure.

### DETAILED DESCRIPTION

For simplicity and illustrative purposes, the present disclosure is described by referring mainly to an exemplary embodiment thereof. In the following description, numerous specific details are set forth in order to provide a thorough understanding of the present disclosure. However, it will be readily apparent to one of ordinary skill in the art that the present disclosure may be practiced without limitation to these specific details.

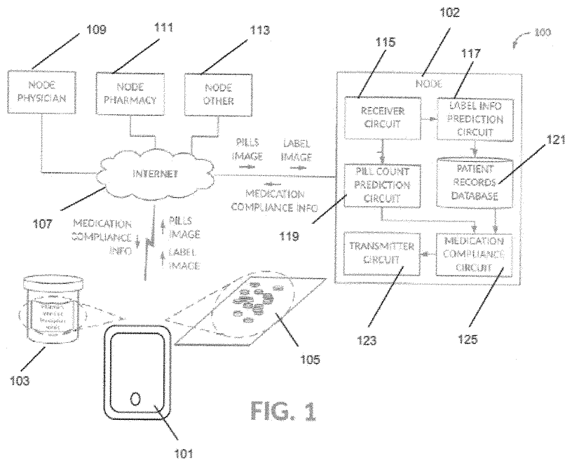

In this disclosure, systems and methods of monitoring medication compliance are provided. For example, FIG. 1 illustrates one embodiment of a system 100 of monitoring medication compliance in conformance with various aspects as described herein. This system includes a node 102 for analyzing and storing patient information and data collected about medications. A hand-held device 101, i.e., a smart phone, tablet, smartwatch, a telehealth or other hand held device which has a camera, microphone or accelerometer, can take a photograph of a pill bottle label 103. The pills 105 from the pill bottle can be spread out on a flat surface and a photograph made of the pills. Images of the label and pills are transmitted via the internet 107 to the node 100 for processing. The node 102 includes a receiver circuit 115 that leads to a label info prediction circuit 117 and a pill count prediction circuit 119. The pill count is analyzed in light of date from the patient records database 121 by the medical compliance circuit 125. A transmitter 123 can transmit information or a warning signal to the patient, a caregiver, a nurse or a physician. Other nodes interacting with the system node 100 or the hand-held device 101 include node physician 109, node pharmacy 111 or node other 113.

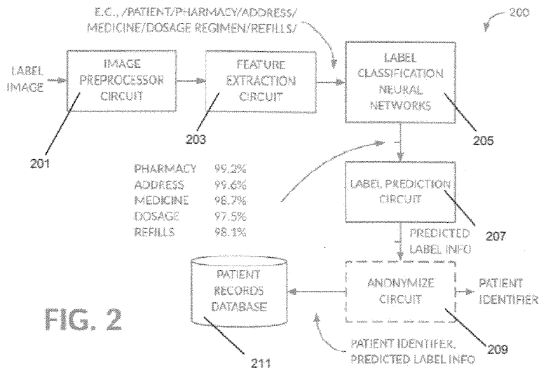

FIG. 2 illustrates one embodiment of a system 200 of predicting medication label information in conformance with various aspects as described herein, which shows the circuit or system 200 that analyzes the label image. The label image first enters the image processor circuit 201. The feature extraction circuit 203 extracts the features from the label where the label information extracts features (such as name, medication, dosage, date pharmacy, etc.) Artificial Intelligence (AI) or Machine Language (ML) classifies the information using label classification neural networks 205. The label prediction circuit 207 assigns probability values to the various factors such as PHARMACY, ADDRES, MEDICINE, DOSAGE and REFILLS. The probability values can be determined using Bayesian inferences. Alternately, convoluted neural networks (CNN) can be used. Since patient data is confidential an anonymize circuit 209 is necessary to protect the patient's data. The data can be encrypted using, for example, Pretty Good Privacy (PGP). Symmetric key or public key can be used. The data is finally entered into the patient records database 211.

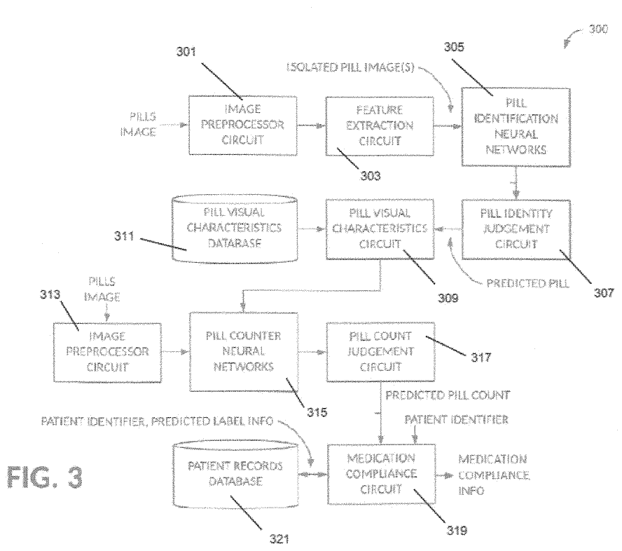

FIG. 3 illustrates one embodiment of a system 300 of predicting a medication pill count in conformance with various aspects as described herein. The pills image is input into an image processor circuit 301. The features of the pills are extracted by the feature extraction circuit 303, which are then processed by the pill identification neural networks 305 using Artificial Intelligence (AI) or Machine Language (ML). The pill identity judgement circuit 307 predicts the pill, and the information is fed to the pill visual characteristics circuit 309 for comparison visual characteristic information stored in the pill visual characteristics database 311. The pills image is also inputted into the image processor circuit 313, where the pill counter neural networks 315 uses AI or ML to evaluate the pill visual characteristics to predict the pill count in the pill count judgment circuit 317. The medication compliance circuit 319 takes this information, identifies the patient and accesses the predicted label information stored in the patient records database 321. The medication compliance circuit 319 then transmits to medication compliance information.

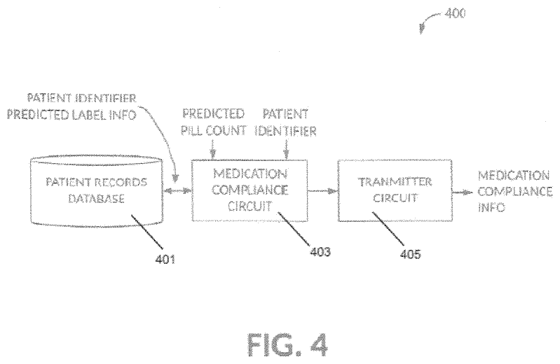

FIG. 4 illustrates one embodiment of a system 400 of determining medication compliance in accordance with various aspects as described herein. The predicted pill count and patient identifier are fed to the medication compliance circuit 403 along with patient identifier predicted label information from the patient records database 401. The AI or ML generated compliance result is then transmitted by the transmitter circuit 405 to the caregiver, nurse or physician, via internet, email, smartphone, text message, etc.

Note that the apparatuses described above may perform the methods herein and any other processing by implementing any functional means, modules, units, or circuitry. In one embodiment, for example, the apparatuses comprise respective circuits or circuitry configured to perform the steps shown in the method figures. The circuits or circuitry in this regard may comprise circuits dedicated to performing certain functional processing and/or one or more microprocessors in conjunction with memory. For instance, the circuitry may include one or more microprocessor or microcontrollers, as well as other digital hardware, which may include digital signal processors (DSPs), special-purpose digital logic, and the like. The processing circuitry may be configured to execute program code stored in memory, which may include one or several types of memory such as read-only memory (ROM), random-access memory, cache memory, flash memory devices, optical storage devices, etc. Program code stored in memory may include program instructions for executing one or more telecommunications and/or data communications protocols as well as instructions for carrying out one or more of the techniques described herein, in several embodiments. In embodiments that employ memory, the memory stores program code that, when executed by the one or more processors, carries out the techniques described herein.

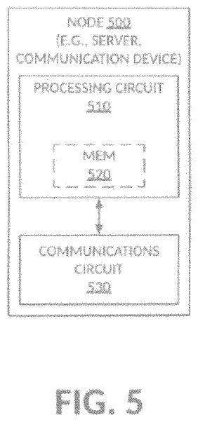

FIG. 5 illustrates one embodiment of a node 500 (e.g., server, communication device, or the like) in accordance with various aspects as described herein. As shown, the node 500 includes processing circuitry 510 and communication circuitry 530. The communication circuitry 530 is configured to transmit and/or receive information to and/or from one or more other nodes, e.g., via any communication technology. The processing circuitry 510 is configured to perform processing described above, such as by executing instructions stored in memory 520. The processing circuitry 510 in this regard may implement certain functional means, units, or modules.

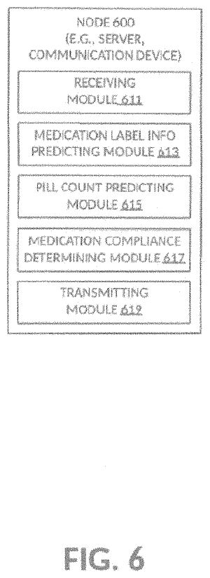

FIG. 6 illustrates another embodiment of a node 600 in accordance with various aspects as described herein. As shown, the node 600 implements various functional means, units, or modules (e.g., via the processing circuitry 510 in FIG. 5, via software code), or circuits. In one embodiment, these functional means, units, modules, or circuits (e.g., for implementing the method(s) herein) may include for instance: a receiving module 611 for receiving a first image of a medication label (e.g., medication, dosage regimen, or the like) disposed on a medication container and a second image of one or more pills distributed on a surface from the medication container; a medication label information predicting unit 613 for predicting information indicated on the medication label based on the first image; a pill count predicting unit 615 for predicting a number of the pills distributed on the surface based on the second image; a medication compliance determining unit 617 for determining information associated with compliance to the dosage regimen of the medication based on the predicted number of pills and the dosage regimen. The information is transmitted via the transmitting module 619.

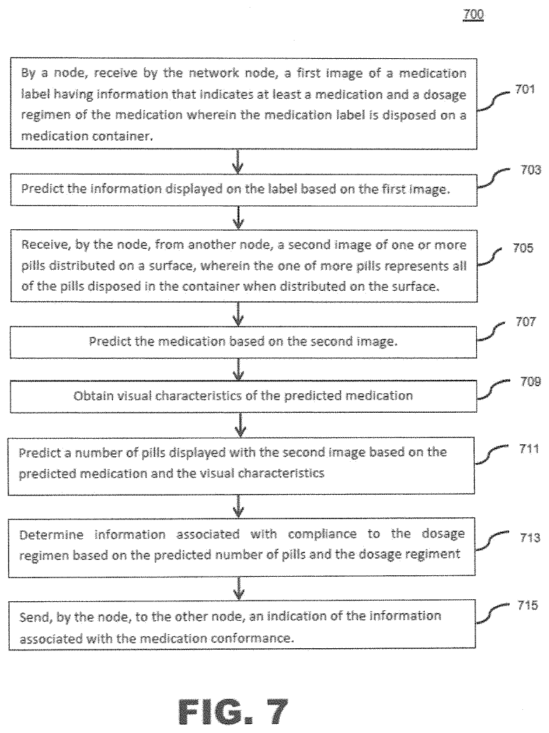

FIG. 7 illustrates one embodiment of a method 700 of monitoring medication compliance in accordance with various aspects as described herein. In step 701 a node, receives by the network node, a first image of a medication label having information that indicates at least a medication and a dosage regiment of the medication. The medication label is disposed on a mediation container. In step 703, the information displayed on the label is predicted based on the first image. In step 705, the node receives, from another node, a second image of one or more pills distributed on a surface, where the one or more pills represents all of the pills disposed in the container when distributed on the surface. In step 707, the medication is predicted based on the second image. In step 709, the visual characteristics of the predicted medication are obtained. In step 711, the number of pills displayed on the second image is predicted, based on the predicted medication and the visual characteristics. Step 713 determines information associated with compliance with the dosage regimen of the medication based on the predicted number of pills and the dosage regiment. Step 715 sends, by the node to the other node, an indication of the information associated with the medication conformance.

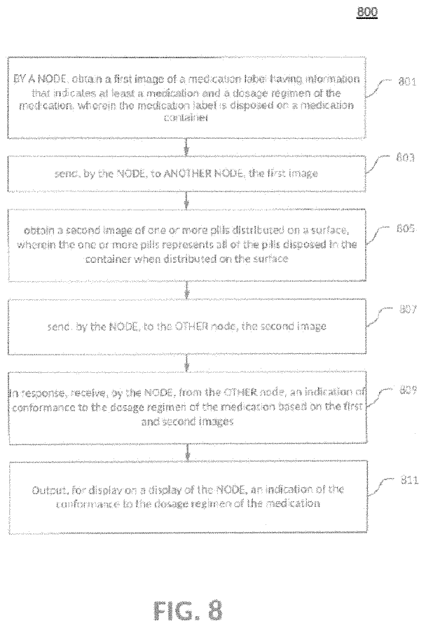

FIG. 8 illustrates another embodiment of a method 800 of monitoring medication compliance in accordance with various aspects as described herein. In step 801, a node obtains a first image of a medication label having information that indicates at least a medication and a dosage regiment of the medication, where the medication label is disposed on a medication container. In step 803, the node sends the first image to another node. In step 805, a second image of one or more pills distributed on a surface is obtained, where the one or more pills represents of the pills disposed in the container when distributed on the surface. In step 807 the node sends the second image to the other node. In step 809, in response, the node receives from the other node, an indication of conformance to the dosage regimen based on the first and second images. Step 811 outputs for display on a display of the node, and an indication of conformance to the dosage regimen of the medication.

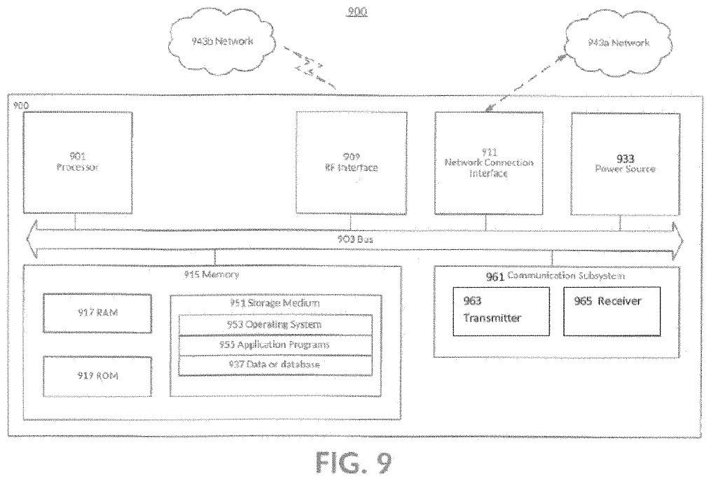

FIG. 9 illustrates another embodiment of a node in accordance with various aspects as described herein. In some instances, the node 900 may be referred as a server, a communication device, a base station, a core node, a handheld computer, a desktop computer, a laptop computer, a tablet computer, a set-top box, a television, an appliance, a medical device, or some other like terminology. In other instances, the node 900 may be a set of hardware components. In FIG. 9, the node 900 may be configured to include a processor 901 that is operatively coupled to a radio frequency (RF) interface 909, a network connection interface 911, which connects via bus 903, a memory 915 including a random access memory (RAM) 917, a read only memory (ROM) 919, a storage medium 931 or the like, a communication subsystem 951, a power source 933, another component, or any combination thereof. The memory 915 may be used to store one or more databases. The memory 915 may include a storage medium 951, an operating system 953, applications programs 955 and at least one database 937 The storage medium 951 may include an operating system 953, an application program 935, data or database 937, or the like. The communication subsystem 961 may include a transmitter 963 and a receiver 965. Specific devices may utilize all of the components shown in FIG. 9, or only a subset of the components, and levels of integration may vary from device to device. Further, specific devices may contain multiple instances of a component, such as multiple processors, memories, transceivers, transmitters, receivers, etc. For instance, a computing device may be configured to include a processor and a memory.

In FIG. 9, the processor 901 may be configured to process computer instructions and data. The processor 901 may be configured as any sequential state machine operative to execute machine instructions stored as machine-readable computer programs in the memory, such as one or more hardware-implemented state machines (e.g., in discrete logic, FPGA, ASIC, etc.); programmable logic together with appropriate firmware; one or more stored-program, general-purpose processors, such as a microprocessor or Digital Signal Processor (DSP), together with appropriate software; or any combination of the above. For example, the processor 901 may include two computer processors. In one definition, data is information in a form suitable for use by a computer. It is important to note that a person having ordinary skill in the art will recognize that the subject matter of this disclosure may be implemented using various operating systems or combinations of operating systems or on the cloud.

In FIG. 9, the RF interface 909 may be configured to provide a communication interface to RF components such as a transmitter, a receiver, and an antenna. The network connection interface 911 may be configured to provide a communication interface to a network 943a. The network 943a may encompass wired and wireless communication networks such as a local-area network (LAN), a wide-area network (WAN), a computer network, a wireless network, a telecommunications network, another like network or any combination thereof. For example, the network 943a may be a Wi-Fi network. The network connection interface 911 may be configured to include a receiver and a transmitter interface used to communicate with one or more other nodes over a communication network according to one or more communication protocols known in the art or that may be developed, such as Ethernet, TCP/IP, SONET, ATM, or the like. The network connection interface 911 may implement receiver and transmitter functionality appropriate to the communication network links (e.g., optical, electrical, and the like). The transmitter and receiver functions may share circuit components, software or firmware, or alternatively may be implemented separately.

In this embodiment, the RAM 917 may be configured to interface via the bus 903 to the processor 901 to provide storage or caching of data or computer instructions during the execution of software programs such as the operating system, application programs, and device drivers. The ROM 919 may be configured to provide computer instructions or data to the processor 901. For example, the ROM 919 may be configured to be invariant low-level system code or data for basic system functions such as basic input and output (I/O), startup, or reception of keystrokes from a keyboard that are stored in a non-volatile memory. The storage medium 931 may be configured to include memory such as RAM, ROM, programmable read-only memory (PROM), erasable programmable read-only memory (EPROM), electrically erasable programmable read-only memory (EEPROM), magnetic disks, optical disks, floppy disks, hard disks, removable cartridges, flash drives. In one example, the storage medium 931 may be configured to include an operating system 933, an application program 935 such as a web browser application, a widget or gadget engine or another application, and a data file 937.

In FIG. 9, the processor 901 may be configured to communicate with a network 943b using the communication subsystem 951. The network 943a and the network 943b may be the same network or networks or different network or networks. The communication subsystem 961 may be configured to include one or more transceivers used to communicate with the network 943b. The one or more transceivers may be used to communicate with one or more remote transceivers of another node or client device according to one or more communication protocols known in the art or that may be developed, such as IEEE 802.xx, CDMA, WCDMA, GSM, LTE, NR, NB IoT, UTRAN, WiMax, or the like.

In another example, the communication subsystem 951 may be configured to include one or more transceivers used to communicate with one or more remote transceivers of another node or client device according to one or more communication protocols known in the art or that may be developed, such as IEEE 802.xx, CDMA, WCDMA, GSM, LTE, NR, NB IoT, UTRAN, WiMax, or the like. Each transceiver may include a transmitter 963 or a receiver 965 to implement transmitter or receiver functionality, respectively, appropriate to the RAN links (e.g., frequency allocations and the like). Further, the transmitter 963 and the receiver 965 of each transceiver may share circuit components, software, or firmware, or alternatively may be implemented separately.

In the current embodiment, the communication functions of the communication subsystem 961 may include data communication, voice communication, multimedia communication, short-range communications such as Bluetooth, near-field communication, location-based communication such as the use of the global positioning system (GPS) to determine a location, another like communication function, or any combination thereof. For example, the communication subsystem 961 may include cellular communication, Wi-Fi communication, Bluetooth communication, and GPS communication. The network 943b may encompass wired and wireless communication networks such as a local-area network (LAN), a wide-area network (WAN), a computer network, a wireless network, a telecommunications network, another like network or any combination thereof. For example, the network 943b may be a cellular network, a Wi-Fi network, and a near-field network. The power source 933 may be configured to provide an alternating current (AC) or direct current (DC) power to components of the node 900.

In FIG. 9, the storage medium 951 may be configured to include a number of physical drive units, such as a redundant array of independent disks (RAID), a floppy disk drive, a flash memory, a USB flash drive, an external hard disk drive, thumb drive, pen drive, key drive, a high-density digital versatile disc (HD-DVD) optical disc drive, an internal hard disk drive, a Blu-Ray optical disc drive, a holographic digital data storage (HDDS) optical disc drive, an external mini-dual in-line memory module (DIMM) synchronous dynamic random access memory (SDRAM), an external micro-DIMM SDRAM, a smartcard memory such as a subscriber identity module or a removable user identity (SIM/RUIM) module, other memory, or any combination thereof. The storage medium 951 may allow the node 900 to access computer-executable instructions, application programs or the like, stored on transitory or non-transitory memory media, to off-load data, or to upload data. An article of manufacture, such as one utilizing a communication system may be tangibly embodied in storage medium 931, which may comprise a computer-readable medium.

The functionality of the methods described herein may be implemented in one of the components of the node 900 or partitioned across multiple components of the node 900. Further, the functionality of the methods described herein may be implemented in any combination of hardware, software or firmware. In one example, the communication subsystem 951 may be configured to include any of the components described herein. Further, the processor 901 may be configured to communicate with any of such components over the bus 903. In another example, any of such components may be represented by program instructions stored in memory that when executed by the processor 901 performs the corresponding functions described herein. In another example, the functionality of any of such components may be partitioned between the processor 901 and the communication subsystem 951. In another example, the non-computative-intensive functions of any of such components may be implemented in software or firmware and the computative-intensive functions may be implemented in hardware.

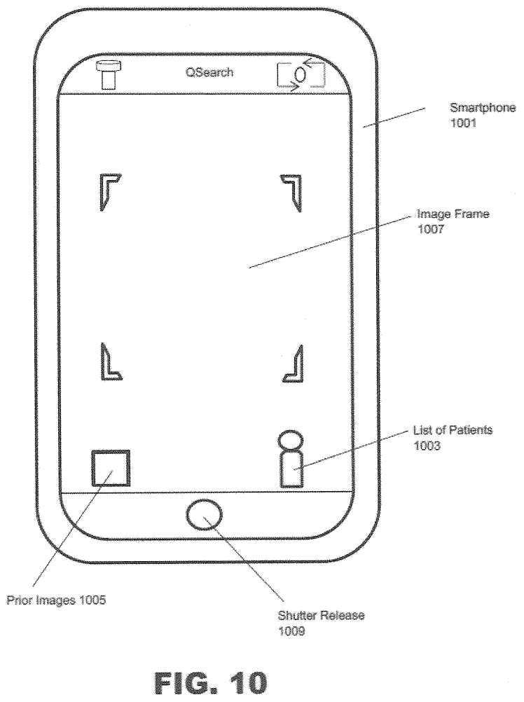

FIG. 10 illustrates an embodiment of the disclosure as implemented as an app on a hand-held device, typically a smartphone 1001. The app includes a list of patients 1003 and a compendium of prior images 1005. A photograph of the pill container or number of pills is taken using the image picture frame 1007 and the shutter release.

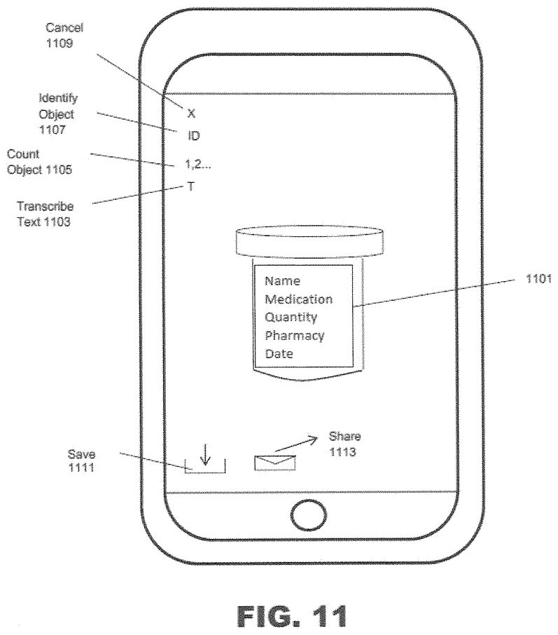

FIG. 11 illustrates an embodiment of the app where a picture is taken of a label of a pill container 1101. Functions of the app include transcribing text 1103, counting objects 1105, identifying objects 1107 and canceling 1109. The photograph and its associated data can either be saved 1111 or shared 1113. The sharing can be with another node or to caretaker, nurse or physician. The saving can be to the local memory on the smartphone or to the memory of the other node or to the cloud. A significant amount of information is contained on the label of the pill container. This information can include the patient name, the manufacturer/brand name, form (e.g., 10 mg tablets), the generic medication name, the directions, the expiration date, refills, physician name, physician phone number, quantity, prescription number, date filled, pharmacy name and address, pharmacy phone number and a barcode.

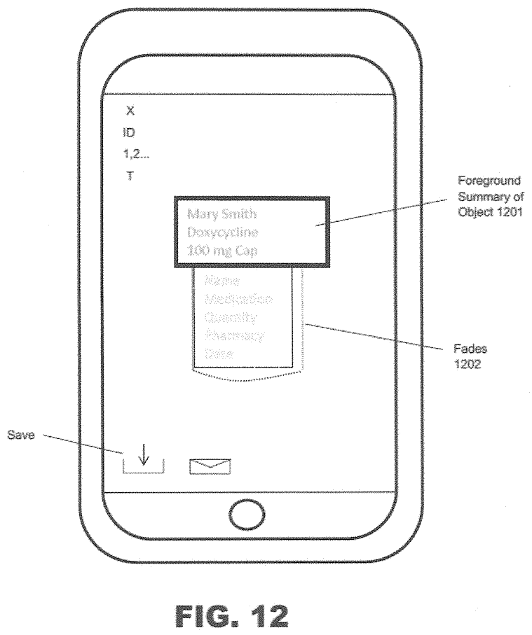

FIG. 12 illustrates the visuals of the smartphone as the information is extracted. In the foreground appears a summary of the object 1201. The picture of the object itself, i.e., the label of the container appears to fade 1202, although the image itself is not lost and can remain in the memory or other associated databases.

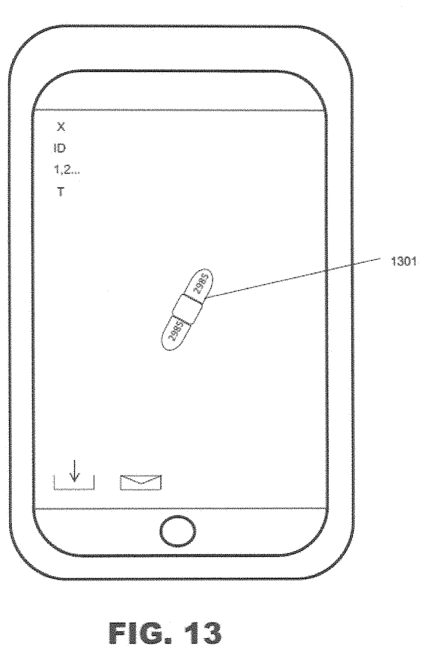

FIG. 13 shows the photograph of a single pill 1301 taken from the container. The photograph of the single pill is analyzed using ML for identity. Identifying factors include, size, shape, color, marks and any characters imprinted or impressed into the pill.

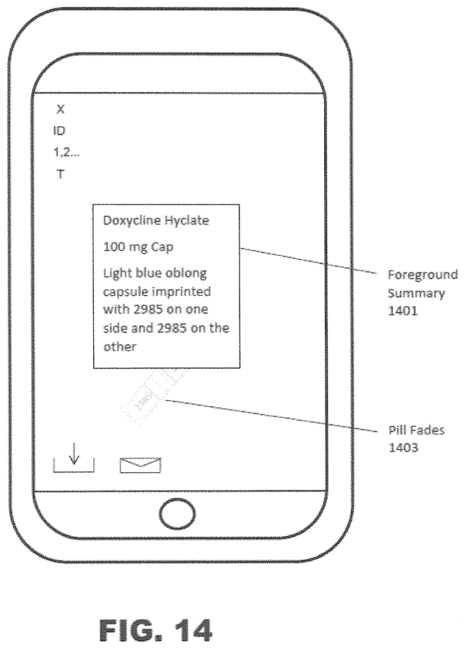

As is illustrated in FIG. 14, the ML analysis of the pill information including name, dosage, appearance, markings etc. appears in the foreground 1401 while the image of the pill fades 1403.

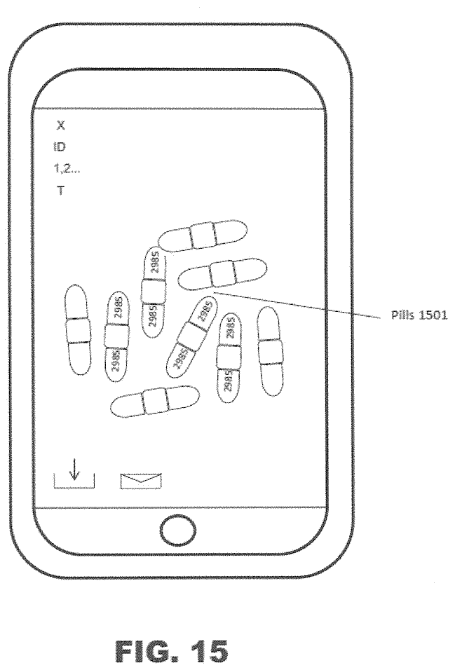

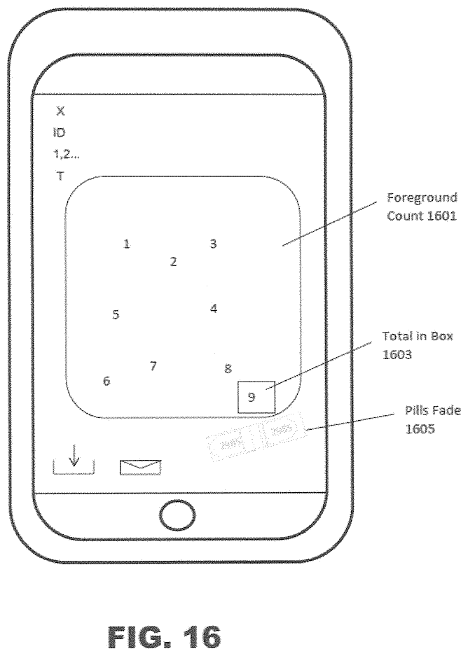

Then the pills have to be counted. The pills are removed from the container and placed on a flat surface. A photograph of all the pills 1501 is taken, as is illustrated in FIG. 15. The app next displays a foreground count of the pills 1601, where each pill is counted. The total number of pills appears in the box 1603 while the image of the pills fades 1605, as is illustrated in FIG. 16.

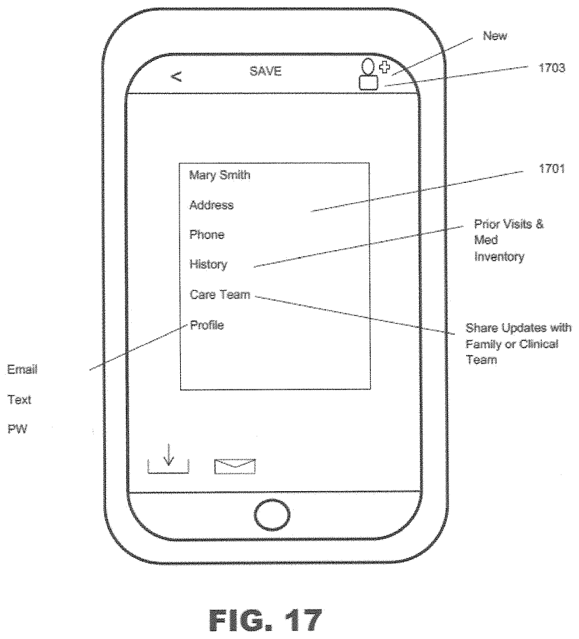

FIG. 17 illustrates the information available through the app. The information 1701 includes the identification of the patient, i.e., name, address, phone number, email, etc., a record of prior visits and medical inventory and the care team. Updates can be shared with the family, clinical team, nurse or physician. The caregiver utilizing the app can also select a new patient or bring up another profile 1703, for the assessment of the next patient.

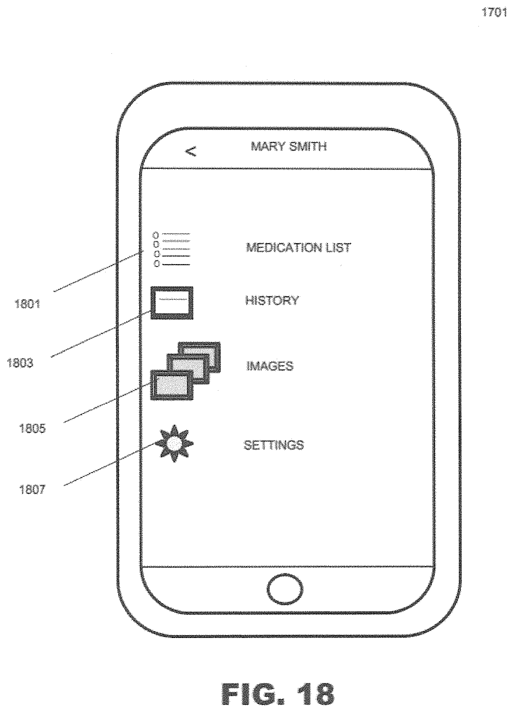

FIG. 18 illustrates menus available through the app. These menus include but are not restricted to medication lists 1801, history 1803, images 1805 and settings 1807.

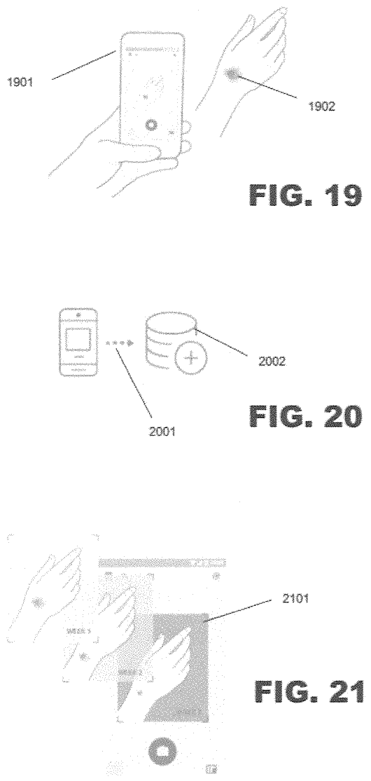

Monitoring of wound healing is another aspect of the disclosure. FIG. 19 illustrates a smartphone 1901 acting as a node taking a photograph of a wound 1902. As is illustrated in FIG. 20, the photograph is transmitted 2001 to another node 2002 where the photograph is analyzed for wound size, shape, color, etc. and compared to previous photographs of the wound and a database of wounds using ML. Information about the progression of wound healing can be relayed back to the smartphone to give a history of the wound 2101, as is shown in FIG. 21. The node can also be a tablet, a smartwatch or other hand held device.

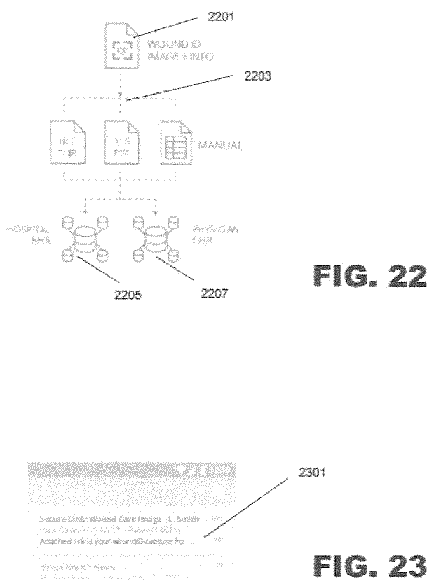

The classification results of the wound can be uploaded to an electronic health record, as is illustrated in FIG. 22. The wound image and information 2201 can be inputted 2203 to a spreadsheet, EHR or manually inputted. The analysis is then output to at least one of the Hospital EHR 2205 or the physician EHR 2207. As is illustrated in FIG. 23, classification results 2301 can be shared with providers and caregivers.

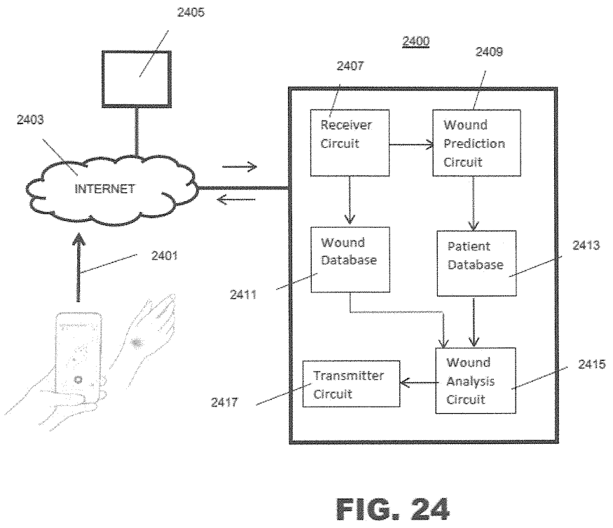

FIG. 24 illustrates an embodiment of the wound healing module of the disclosure. Wound image 2401 is transmitted to node 2400 via the internet 2403. Other nodes 2405 can include a physician node, a nurse node or a pharmacy node. The node 2400 includes a receiver circuit 2407 which transmits the image to the wound prediction circuit 2409, which uses ML to predict the wound type and characteristics. The wound prediction circuit 2409 sends the predicted wound to the patient database 2413, which contains patient information, medications and the wound history. The image is also sent to the wound database 2411, which is a compendium of wound analysis. The wound analysis circuit 2415 uses ML to generate a wound profile, which is transmitted to the physician, nurse, pharmacist, etc. via the transmission circuit 2417. An alert can be given if remedial action is necessary, such as changing a dressing, adjusting medication, etc.

The disclosure offers many advantages. AI or ML reduces inventory time from 6 minutes to about 1 minute per medication. In-home inventory captures a complete list of prescription, over the counter, herbals, vitamins and supplements. The technology can impute days on hand and adherence based upon the last fill date, refills and prior visit history.

The disclosure of the is utilized in the context of Chronic Care Management (CCM). CCM is overseen by a medical provider. This can be an physician (MD), doctor of osteopathy (DO), an advanced practice registered nurse (APRN), a nurse practitioner (NP), physician assistant (PA), a clinical nurse specialist (CNS) or a certified nurse-midwife (CNM). The disclosure permits efficient CCM, where an approximately 20 minute visit by a certified nursing assistant (CNA) or a nurse can have consistent and impactful face-to-face interaction and data collection. Remote office-based RNs or MDs evaluate data, images and notes captured during the visits, reviews medications and vitals, and approve care plans supporting optimal health. The visit documentation includes reconciled medication list, sentiment and vitals, and are shared electronically with the practice and the patient. ML evaluates the key medication data and vital signs, organizes for RN or physician review and highlights potential problems.

The CCM services during a visit include a vitals check, a sentiment check, a medication inventory, an up-to-date medication list and medication reconciliation. A longer visit can include a medication schedule, and adherence check and patient portal login assistance.

The technology of the disclosure permits building an accurate list of client medications. Inventory all prescription, over the counter, vitamins and supplements. Pinpoint duplicate therapies, spot contraindications, and highlight medication that increase fall risk. Everything is automated and in one package. The wound module permits monitoring of pressure ulcers and bed sores consistently, tracking the healing progress, and identifying potential infections. Stage the wound, identify infected skin, and see a time lapse view of the healing process. Identify related medication and mobility factors that impact wound healing.

Although the disclosure uses smartphones, computers and AI/ML, the result is a technology that is integrated into a practical application.

The previous detailed description is merely illustrative in nature and is not intended to limit the present disclosure, or the application and uses of the present disclosure. Furthermore, there is no intention to be bound by any expressed or implied theory presented in the preceding field of use, background, summary, or detailed description. The present disclosure provides various examples, embodiments and the like, which may be described herein in terms of functional or logical block elements. The various aspects described herein are presented as methods, devices (or apparatus), systems, or articles of manufacture that may include a number of components, elements, members, modules, nodes, peripherals, or the like. Further, these methods, devices, systems, or articles of manufacture may include or not include additional components, elements, members, modules, nodes, peripherals, or the like.

Furthermore, the various aspects described herein may be implemented using standard programming or engineering techniques to produce software, firmware, hardware (e.g., circuits), or any combination thereof to control a computing device to implement the disclosed subject matter. It will be appreciated that some embodiments may be comprised of one or more generic or specialized processors such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the methods, devices and systems described herein. Alternatively, some or all functions could be implemented by a state machine that has no stored program instructions, or in one or more application specific integrated circuits (ASICs), in which each function or some combinations of certain of the functions are implemented as custom logic circuits. Of course, a combination of the two approaches may be used. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

The term “article of manufacture” as used herein is intended to encompass a computer program accessible from any computing device, carrier, or media. For example, a computer-readable medium may include: a magnetic storage device such as a hard disk, a floppy disk or a magnetic strip; an optical disk such as a compact disk (CD) or digital versatile disk (DVD); a smart card; and a flash memory device such as a card, stick or key drive. Additionally, it should be appreciated that a carrier wave may be employed to carry computer-readable electronic data including those used in transmitting and receiving electronic data such as electronic mail (e-mail) or in accessing a computer network such as the Internet or a local area network (LAN). Of course, a person of ordinary skill in the art will recognize many modifications may be made to this configuration without departing from the scope or spirit of the subject matter of this disclosure.

Throughout the specification and the embodiments, the following terms take at least the meanings explicitly associated herein, unless the context clearly dictates otherwise. Relational terms such as “first” and “second,” and the like may be used solely to distinguish one entity or action from another entity or action without necessarily requiring or implying any actual such relationship or order between such entities or actions. The term “or” is intended to mean an inclusive “or” unless specified otherwise or clear from the context to be directed to an exclusive form. Further, the terms “a,” “an,” and “the” are intended to mean one or more unless specified otherwise or clear from the context to be directed to a singular form. The term “include” and its various forms are intended to mean including but not limited to. References to “one embodiment,” “an embodiment,” “example embodiment,” “various embodiments,” and other like terms indicate that the embodiments of the disclosed technology so described may include a particular function, feature, structure, or characteristic, but not every embodiment necessarily includes the particular function, feature, structure, or characteristic. Further, repeated use of the phrase “in one embodiment” does not necessarily refer to the same embodiment, although it may. The terms “substantially,” “essentially,” “approximately,” “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. A device or structure that is “configured” in a certain way is configured in at least that way, but may also be configured in ways that are not listed.

What is claimed is:

1. A method performed by a first node of monitoring medication compliance, comprising:
receiving, by a first node, from a second node, a first image of a medication label having information that indicates at least a medication and a dosage regimen of the medication, wherein the medication label is disposed on a medication container;
predicting the information displayed on the label based on the first image;
receiving, by the first node, from the second node, a second image of one or more pills distributed on a surface, wherein the one or more pills represents all of the pills disposed in the container when distributed on the surface;
predicting the medication based on the second image;
obtaining visual characteristics of the predicted medication;
predicting a number of pills displayed in the second image based on the predicted medication and the visual characteristics;
determining information associated with compliance to the dosage regimen of the medication based on the predicted number of pills and the dosage regimen; or
sending, by the first node, to the second node, an indication of the information associated with the medication compliance.

2. The method of claim 1, wherein the second node is a wireless device.

3. The method of claim 1, wherein communication between the first node and the second node is via a network.

4. The method of claim 1, wherein the first node is a server.

5. The method of claim 1, wherein said sending is so that the second node is operable to display the indication of the information associated with the medication compliance.

6. The method of claim 1, wherein the first image is processed by an image preprocessor circuit, a feature extraction circuit, label classification neural networks, and a label prediction circuit.

7. The method of claim 1, further comprising:
anonymizing the predicted label information to obtain a patient identifier associated with the anonymized label information; and
storing the patient identifier and the anonymized label information in an anonymized patient records database.

8. The method of claim 1, wherein said predicting the medication includes:
extracting one or more isolated pill images from the second image; and
determining which one of a plurality of predetermined pill images is closest in visual characteristics to the one or more isolated pill images to obtain the predicted medication.

9. The method of claim 7, wherein the second image is processed by a pill counter neural networks so as to obtain a predicted pill count.

10. A first node, comprising:
at least one processor and a memory, the memory comprising instructions executable by the at least one processor whereby the first node is configured to:
receive, from a second node, a first image of a medication label having information that indicates at least a medication and a dosage regimen of the medication, wherein the medication label is disposed on a medication container;
predict the information displayed on the label based on the first image;
receive, from the second node, a second image of one or more pills distributed on a surface, wherein the one or more pills represents all of the pills disposed in the container when distributed on the surface;
predict the medication based on the second image;
obtain visual characteristics of the predicted medication;
predict a number of pills displayed in the second image based on the predicted medication and the visual characteristics;
determine information associated with compliance to the dosage regimen of the medication based on the predicted number of pills and the dosage regimen; and
send, to the second node, an indication of the information associated with the medication compliance.

11. A method performed by a second node of monitoring medication compliance, comprising:
obtaining a first image of a medication label having information that indicates at least a medication and a dosage regimen of the medication, wherein the medication label is disposed on a medication container;
sending, by the second node, to a first node, the first image;
obtaining a second image of one or more pills distributed on a surface, wherein the one or more pills represents all of the pills disposed in the container when distributed on the surface; and
receiving, by the second node, from the first node, an indication of compliance to the dosage regimen of the medication based on the first and second images responsive to sending, by the second node, to the first node, the second image.

12. The method of claim 11, further comprising:
outputting, for display on a display of the first node, an indication of the medication compliance.

13. The method of claim 11, wherein the second node is a wireless device.

14. The method of claim 11, wherein the second node includes a receiver circuit, a label information prediction circuit, a pill count prediction circuit, a patient records database, a medication compliance circuit and a transmitter circuit.

15. The method of claim 11, wherein the first image is processed by an image preprocessor circuit, a feature extraction circuit, label classification neural networks, and a label prediction circuit.

16. The method of claim 15, wherein the label prediction circuit is configured to output predicted label information to an anonymize circuit, which transmits the patient identifier and the predicted label information to a patient records database.

17. The method of claim 11, wherein the second image is processed by an image preprocessor circuit and a feature extraction circuit, which outputs isolated pill images to pill identification neural networks and a pill identity judgement circuit, which outputs the predicted pill to a pill visual characteristics circuit, which compares the predicted pill with data from a pill visual characteristics database.

18. The first node of claim 17, wherein the second image is processed by a pill counter neural network and a pill judgement circuit, to output a predicted pill count and the predicted pill to a medication compliance circuit, which interacts with the patient records database to output the information associated with the medication compliance.

19. A second node, comprising:
at least one processor and a memory, the memory comprising instructions executable by the at least one processor whereby the second node is configured to:
obtain a first image of a medication label having information that indicates at least a medication and a dosage regimen of the medication, wherein the medication label is disposed on a medication container;
send, to a first node, the first image;
obtain a second image of one or more pills distributed on a surface, wherein the one or more pills represents all of the pills disposed in the container when distributed on the surface;
send, to the first node, the second image; and
receive, from the first node, an indication of compliance to the dosage regimen of the medication based on the first and second images.

20. A system for monitoring medication compliance, comprising:
a first node including a camera, a microphone, an accelerometer and a memory;
a second node including a memory and a processor;
wherein the first node is configured to obtain a first image of a medication label having information that indicates at least a medication and a dosage regimen of the medication, wherein the medication label is disposed on a medication container, and the first node sends the first image to the second node, and the first node obtains a second image of one or more pills distributed on a surface, wherein the one or more pills represents all of the pills disposed in the container when distributed on the surface; and
wherein the second node is configured to predict information on the label based in the first image, predict the medication based on the second image, obtain visual characteristics of the predicted medication, predict a number of pills displayed in the second image based on the predicted medication and the visual characteristics, determine information associated with compliance to the dosage regimen of the medication based on the predicted number of pills and the dosage regimen, and send to the first node an indication of the information associated with the medication compliance.
